(19) United States
(12) Patent Application Publication
(10) Pub. No.: US 2008/0103170 A1
(43) Pub. Date: May 1, 2008

(54) Nicotinic Acetylcholine Receptor Ligands

(75) Inventors: Robert Jacobs, Research Triangle Park, NC (US); Richard J. Schmiesing, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/721,483

(22) PCT Filed: Dec. 14, 2005
(86) PCT No.: PCT/SE05/01919

§ 371 Date: Jun. 12, 2007

Related U.S. Application Data
(60) Provisional application No. 60/636,680, filed on Dec. 16, 2004.

(51) Int. Cl. A61K 31/4375; A61P 1/04; A61P 25/16; A61P 25/28; A61P 25/34; C07D 453/02

(52) U.S. Cl. 514/305; 546/133

(57) ABSTRACT

Acetylcholine receptor ligands of formula I

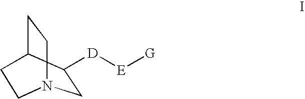

wherein D, E and G are as described in the specification, diastereoisomers, enantiomers, pharmaceutically-acceptable salts, methods of making, pharmaceutical compositions containing, and methods for using the same.

### FIELD OF THE INVENTION

This invention relates to azole compounds or pharmaceutically-acceptable salts thereof, processes for preparing them, pharmaceutical compositions containing them and their use in therapy. The invention also relates to compounds that are ligands for nicotinic acetylcholine receptors (nAChRs).

### BACKGROUND OF THE INVENTION

The use of compounds which bind nicotinic acetylcholine receptors in the treatment of a range of disorders involving reduced cholinergic function such as Alzheimer's disease, cognitive or attention disorders, anxiety, depression, smoking cessation, neuroprotection, schizophrenia, analgesia, Tourette's syndrome, and Parkinson's disease has been discussed in McDonald et al. (1995) “Nicotinic Acetylcholine Receptors: Molecular Biology, Chemistry and Pharmacology”, Chapter 5 in Annual Reports in Medicinal Chemistry, vol. 30, pp. 41-50, Academic Press Inc., San Diego, Calif.; and in Williams et al. (1994) “Neuronal Nicotinic Acetylcholine Receptors,” Drug News & Perspectives, vol. 7, pp. 205-223.

### DESCRIPTION OF THE INVENTION

This invention concerns nicotinic acetylcholine receptor-active compounds according to formula I:

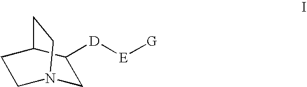

wherein:

D is selected from moieties according to formula II, III, IV or V

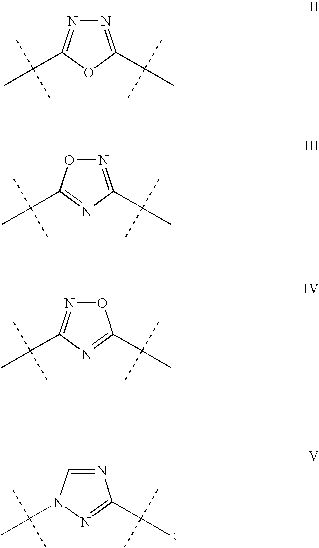

E and G are independently selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;

wherein E and G are unsubstituted or independently have 1, 2 or 3 substituents selected from —C₁-C₆alkyl, —C₂-C₆alkenyl, —C₂-C₆alkynyl, halogen, —CN, —NO₂, —CF₃, —CONR¹R², —S(O)ₙR¹, —NR²R³, —CH₂NR²R³, —OR¹, —CH₂OR¹ or —CO₂R⁴;

R¹, R² and R³ are independently selected at each occurrence from hydrogen, halogen, —C₁-C₄alkyl, aryl, heteroaryl, —C(O)R⁴, —C(O)NHR⁴, —CO₂R⁴ or —SO₂R⁴, or

R² and R³ in combination is —(CH₂)ⱼG(CH₂)ₖ— wherein G is oxygen, sulfur, NR⁴, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl.

The invention also encompasses stereoisomers, enantiomers, in vivo-hydrolysable precursors and pharmaceutically-acceptable salts of compounds of formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Compound of the invention are those according to formula I:

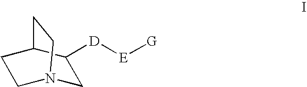

wherein:

D is selected from moieties according to formula II, III, IV or V

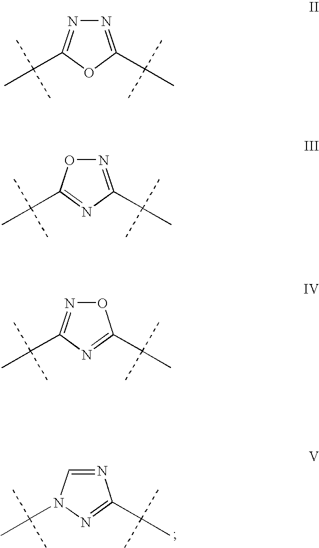

E and G are independently selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;

wherein E and G are unsubstituted or independently have 1, 2 or 3 substituents selected from —C₁-C₆alkyl, —C₂-C₆alkenyl, —C₂-C₆alkynyl, halogen, —CN, —NO₂, —CF₃, —CONR¹R², —S(O)ₙR¹, —NR²R³, —CH₂NR²R³, —OR¹, —CH₂OR¹ or —CO₂R⁴;

R¹, R² and R³ are independently selected at each occurrence from hydrogen, halogen, —C₁-C₄alkyl, aryl, heteroaryl, —C(O)R⁴, —C(O)NHR⁴, —CO₂R⁴ or —SO₂R⁴, or

R² and R³ in combination is —(CH₂)ⱼG(CH₂)ₖ— wherein G is oxygen, sulfur, NR⁴, or a bond;

j is 2, 3 or 4;

k is 0, 1 or 2;

n is 0, 1 or 2, and

R⁴ is independently selected at each occurrence from hydrogen, —C₁-C₄alkyl, aryl, or heteroaryl,

and stereoisomers, enantiomers, in vivo-hydrolysable precursors and pharmaceutically-acceptable salts thereof.

Particular compounds are those of formula I wherein:

E is a 5-membered aromatic or heteroaromatic ring having 1 nitrogen atom and 1 oxygen atom, 1 oxygen atom, or 1 sulfur atom;

G is a 6-membered aromatic ring having 0 or 1 nitrogen atoms;

wherein G is unsubstituted or has 1 substituent selected from —C₁-C₆alkyl, —C₂-C₆alkenyl, —C₂-C₆alkynyl, halogen, —CN, —NO₂, —CF₃, —CONR¹R², —S(O)ₙR¹, —NR²R³, —CH₂NR²R³, —OR¹, —CH₂OR¹ or —CO₂R⁴.

More particular compounds are those of formula I wherein:

E is a 5-membered aromatic or heteroaromatic ring having 1 nitrogen atom and 1 oxygen atom, 1 oxygen atom, or 1 sulfur atom, and

G is a 6-membered aromatic ring having 0 or 1 nitrogen atoms;

wherein G is unsubstituted or has 1 substituent selected from —C₁-C₆alkyl, —CONR¹R² or —NR²R³.

Particular compounds of the invention are those described herein and pharmaceutically-acceptable salts thereof.

In a further aspect the invention encompasses compounds according to formula I wherein one or more of the atoms is a radioisotope of the same element. In a particular form of this aspect of the invention the compound of formula I is labeled with tritium. Such radio-labeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds of the invention labeled with tritium are useful for the discovery of novel medicinal compounds which bind to and modulate the activity, by agonism, partial agonism, or antagonism, of the α7 nicotinic acetylcholine receptor. Such tritium-labeled compounds may be used in assays that measure the displacement of a such compounds to assess the binding of ligand that bind to α7 nicotinic acetylcholine receptors.

In another aspect the invention relates to compounds according to formula I and their use in therapy and to compositions containing them.

In another aspect the invention encompasses the use of compounds according to formula I for the therapy of diseases mediated through the action of nicotinic acetylcholine receptors. A more particular aspect of the invention relates to the use of compounds of formula I for the therapy of diseases mediated through the action of α7 nicotinic acetylcholine receptors.

Another aspect of the invention encompasses a method of treatment or prophylaxis of diseases or conditions in which activation of the α7 nicotinic receptor is beneficial which method comprises administering a therapeutically-effective amount of a compound of the invention to a subject suffering from said disease or condition.

One embodiment of this aspect of the invention is a method of treatment or prophylaxis, wherein the disorder is anxiety, schizophrenia, mania or manic depression.

Another embodiment of this aspect of the invention is a method of treatment or prophylaxis of neurological disorders, psychotic disorders or intellectual impairment disorders, which comprises administering a therapeutically effective amount of a compound of the invention.

Another embodiment of this aspect of the invention is a method of treatment or prophylaxis, wherein the disorder is Alzheimer's disease, learning deficit, cognition deficit, attention deficit, memory loss, or Attention Deficit Hyperactivity Disorder.

Another embodiment of this aspect of the invention is a method of treatment or prophylaxis, wherein the disorder is Parkinson's disease, Huntington's disease, Tourette's syndrome, or neurodegenerative disorders in which there is loss of cholinergic synapses.

Another embodiment of this aspect of the invention is a method of treatment or prophylaxis of jetlag, nicotine addiction, craving, pain, and for ulcerative colitis, which comprises administering a therapeutically effective amount of a compound of the invention.

Yet another embodiment of this aspect of the invention is a method for inducing the cessation of smoking which comprises administering an effective amount of a compound of the invention.

Another embodiment of this aspect of the invention is a pharmaceutical composition comprising a compound of the invention and a pharmaceutically-acceptable diluent, lubricant or carrier.

A further aspect of the invention relates to a pharmaceutical composition useful for treating or preventing a condition or disorder mentioned herein arising from dysfunction of nicotinic acetylcholine receptor neurotransmission in a mammal, preferably a human, comprising an amount of a compound of formula I, an enantiomer thereof or a pharmaceutically-acceptable salt thereof, effective in treating or preventing such disorder or condition, and pharmaceutically-acceptable additives carrier.

Another embodiment of this aspect of the invention relates to use of a pharmaceutical composition of the invention for the treatment, amelioration or prophylaxis of human diseases or conditions in which activation of the α7 nicotinic receptor is beneficial.

Another embodiment of this aspect of the invention is the use of the pharmaceutical composition of the invention for the treatment or prophylaxis of neurological disorders, psychotic disorders or intellectual impairment disorders.

Another embodiment of this aspect of the invention is the use of the pharmaceutical composition of the invention for the treatment or prophylaxis of Alzheimer's disease, learning deficit, cognition deficit, attention deficit, memory loss, Attention Deficit Hyperactivity Disorder, anxiety, schizophrenia, or mania or manic depression, Parkinson's disease, Huntington's disease, Tourette's syndrome, neurodegenerative disorders in which there is loss of cholinergic synapse, jetlag, cessation of smoking, nicotine addiction including that resulting from exposure to products containing nicotine, craving, pain, and for ulcerative colitis.

A further aspect of the invention is the use of a compound according to the invention, an enantiomer thereof or a pharmaceutically-acceptable salt thereof, in the manufacture of a medicament for the treatment or prophylaxis of the diseases or conditions mentioned herein.

Another embodiment of this aspect of the invention is the use of a compound of the invention in the manufacture of a medicament for the treatment or prophylaxis of human diseases or conditions in which activation of the α7 nicotinic receptor is beneficial.

Another embodiment of this aspect of the invention is the use of a compound of the invention in the manufacture of a medicament for the treatment or prophylaxis of neurological disorders, psychotic disorders or intellectual impairment disorders.

Another embodiment of this aspect of the invention is the use of a compound of the invention in the manufacture of a medicament for treatment or prophylaxis of Alzheimer's disease, learning deficit, cognition deficit, attention deficit, memory loss or Attention Deficit Hyperactivity Disorder.

Another embodiment of this aspect of the invention is the use of a compound of the invention in the manufacture of a medicament for treatment or prophylaxis of anxiety, schizophrenia, or mania or manic depression.

Another embodiment of this aspect of the invention is the use of a compound of the invention in the manufacture of a medicament for treatment or prophylaxis of Parkinson's disease, Huntington's disease, Tourette's syndrome, or neurodegenerative disorders in which there is loss of cholinergic synapses.

Another embodiment of this aspect of the invention is the use of a compound as described above in the manufacture of a medicament for the treatment or prophylaxis of jetlag, pain, or ulcerative colitis.

Another aspect of the invention relates to the use of a compound of the invention in the manufacture of a medicament for facilitating the cessation of smoking or the treatment of nicotine addiction or craving including that resulting from exposure to products containing nicotine.

Another aspect of the invention relates to the use of a compound of the invention in combination with other therapeutically-active compounds or substances in pharmaceutical compositions or formulations, methods to treat diseases and conditions, uses as medicaments and uses in the manufacture of medicaments. Particular embodiments of this aspect of the invention comprise other therapeutically-active compounds or substances selected from sedatives, hypnotics, anxiolytics, antipsychotics, antianxiety agents, tranquilizers, and the like.

For the uses, methods, medicaments and pharmaceutical compositions mentioned herein the amount of compound used and the dosage administered will, of course, vary with the compound employed, the mode of administration and the treatment desired. However, in general, satisfactory results are obtained when the compounds of the invention are administered at a daily dosage of from about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

The compounds of formula I, an enantiomer thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier.

Examples of diluents, lubricants and carriers are:

for tablets and dragees: lactose, starch, talc, stearic acid;

for capsules: tartaric acid or lactose;

for injectable solutions: water, alcohols, glycerin, vegetable oils;

for suppositories: natural or hardened oils or waxes.

There is also provided a process for the preparation of such a pharmaceutical composition which process comprises mixing the ingredients.

Compounds according to the invention are agonists of nicotinic acetylcholine receptors. While not being limited by theory, it is believed that agonists of the α7 nicotinic acetylcholine receptor (nAChR) subtype are useful in the treatment or prophylaxis of neurological disorders, psychotic disorders and intellectual impairment disorders, and to have advantages over compounds which are or are also agonists of the α4 nAChR subtype. Therefore, compounds which are selective for the α7 nAChR subtype are preferred. The compounds of the invention are indicated as pharmaceuticals, in particular in the treatment or prophylaxis of neurological disorders, psychotic disorders and intellectual impairment disorders. Examples of psychotic disorders include schizophrenia, mania and manic depression, and anxiety. Examples of intellectual impairment disorders include Alzheimer's disease, learning deficit, cognition deficit, attention deficit, memory loss, and Attention Deficit Hyperactivity Disorder. The compounds of the invention may also be useful as analgesics in the treatment of pain, chronic pain, and in the treatment or prophylaxis of Parkinson's disease, Huntington's disease, Tourette's syndrome, and neurodegenerative disorders in which there is loss of cholinergic synapses.

Compounds of the invention may further useful for the treatment or prophylaxis of jetlag, for use in inducing the cessation of smoking, craving, and for the treatment or prophylaxis of nicotine addiction including that resulting from exposure to products containing nicotine.

It is also believed that compounds according to the invention are useful in the treatment and prophylaxis of ulcerative colitis.

The compounds of the invention have the advantage that they may be less toxic, be more efficacious, be longer acting, have a broader range of activity, be more potent, produce fewer side effects, are more easily absorbed or have other useful pharmacological properties.

The compounds of formula I exist in tautomeric or enantiomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

As used herein, unless otherwise indicated, “C₁₋₄alkyl” includes but is not limited to methyl, ethyl, n-propyl, n-butyl, i-propyl, i-butyl, t-butyl, s-butyl moieties, whether alone or part of another group, C₁₋₄alkyl groups may be straight-chained or branched, and C₃₋₄ alkyl groups include the cyclic alkyl moieties cyclopropyl and cyclobutyl.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein, unless otherwise indicated, aryl refers to a phenyl ring which may have 1, 2 or 3 substituents selected from: halogen, C₁₋₄alkyl, C₂₋₄alkenyl, C₂₋₄alkynyl, C₁₋₄alkyl, CN, NO₂, and CF₃.

As used herein, unless otherwise indicated, heteroaryl refers to a 5- or 6-membered aromatic or heteroaromatic ring having 1, 2 or 3 heteroatoms selected from nitrogen oxygen and sulfur, provided that heteroaromatic rings contains at least one nitrogen, oxygen, or sulfur atom.

As used herein, unless otherwise indicated, halogen refers to fluorine, chlorine, bromine, or iodine.

Where necessary, hydroxy, amino, or other reactive groups may be protected using a protecting group as described in the standard text “Protecting groups in Organic Synthesis”, 3^{rd} Edition (1999) by Greene and Wuts.

Unless otherwise stated, reactions are conducted under an inert atmosphere, preferably under a nitrogen atmosphere and are usually conducted at a pressure of about one to about three atmospheres, preferably at ambient pressure (about one atmosphere).

The compounds of the invention and intermediates may be isolated from their reaction mixtures by standard techniques.

Acid addition salts of the compounds of formula I which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

The compounds of formula I exist in tautomeric or enantiomeric forms, all of which are included within the scope of the invention. The various optical isomers may be isolated by separation of a racemic mixture of the compounds using conventional techniques, e.g. fractional crystallisation, or chiral HPLC. Alternatively the individual enantiomers may be made by reaction of the appropriate optically active starting materials under reaction conditions which will not cause racemisation.

### Pharmacology

The pharmacological activity of compounds of the invention may be measured by using tests such as those set out below:

### Assay for Affinity at an α₇ nAChR Receptor by Measuring the Binding of ¹²⁵I-α-bungarotoxin (BTX) Binding to Rat Hippocampal Membranes

Rat brain cell membranes bearing α₇ nAChR receptors may be prepared by homogenizing hippocampus tissue in 20 volumes of cold homogenization buffer (HB): mM concentrations of HB constituents: tris(hydroxymethyl)aminomethane 50; CaCl₂ 2; MgCl₂ 1; NaCl 120; KCl 5: pH 7.4). Homogenates are centrifuged for 5 minutes at 1000×g, the supernatant saved and the pellets re-extracted and centrifuged. Pooled supernatants are and centrifuged for 20 minutes at 12000×g, the pelleted membranous material is washed, and re-suspended in HB. Membranes (30-150 μg) are incubated with 3 nM [¹²⁵I]α-BTX, 1 mg/mL bovine serum albumin (BSA), together with test compounds in HB for 2 hours at room temperature with gentle shaking. Membranes may then be trapped on Whatman glass fiber filters (thickness C or B) using a Brandel cell harvester and washed 4 times. Pre-treating the filters for 3 hours with 1% (BSA/0.01% PEI (polyethyleneimine) in water will yield low filter blanks (0.07% of total counts per minute). Non-specific binding may be determined by 100 μM (−)-nicotine. Typically specific binding is about 75%.

### Assay for Affinity at Human α₇ nAChR Receptor by Measuring the Binding of ¹²⁵I-α-bungarotoxin (BTX) Binding to Membranes

Membranes may be prepared from HEK cells expressing human α₇ receptors by isolating a 500-40000×g membrane fraction. Such membranes may be used as described for rat brain membranes to assess the binding of compounds to human α₇ receptors.

### Assay for Affinity at an α₄ nAChR Receptor by Measuring the Binding of [³H]-(−)-nicotine Binding to Rat Cortical Membranes

A procedure modified from Martino-Barrows and Kellar (Mol Pharm (1987) 31:169-174) may be used. Rat cortical brain membranes are prepared as described in the [¹²⁵I]α-BTX binding assay, except that a 500-12000×g membrane fraction is prepared. Membranes (30-150 μg) are incubated with 30-100 pM of the epibatidine analog [¹²⁵I]-IPH ((+/−)-exo-2-(2-iodo-5-pyridyl)-7-azabicyclo-[2,2,1]heptane) together with test compound in HB for 1 hour at room temperature with gentle shaking. Membranes may be recovered as described for the [¹²⁵I]α-BTX binding assay. Non-specific binding may be determined by 100 μM carbachol. Typically specific binding is about 84%.

### Analysis of Binding Data Obtained in α₇ nAChR or α₄ nAChR Receptor Assays

IC₅₀ values and pseudo Hill coefficients (n_{H}) may be calculated using the non-linear curve fitting program ALLFIT (DeLean A, Munson P J and Rodbard D (1977) Am. J. Physiol., 235:E97-E102). Saturation curves may be fitted to a one site model, using the non-linear regression program ENZFITTER (Leatherbarrow, R. J. (1987)), yielding a Kd value for [¹²⁵ I]-α-BTX binding to rat α7 nAChR of 1.7 nM and a Kd value for [¹²⁵I]-IPH binding to the rat α4 nAChR of 64 pM. Kᵢ values may be estimated using the general Cheng-Prusoff equation:

Kᵢ=[IC₅₀]/((2+ligand/K_{D})ⁿ)^{1/n}−1)

where a value of n=1 is used whenever n_{H}<1.5 and a value of n=2 is used when n_{H}≧1.5. To account for variability, assays may be performed in triplicate and variability will typically be ±5%. Kᵢ values may be determined using six to 11 drug concentrations.

Compounds of the invention expected to have useful therapeutic activity will be found to have binding affinities (Kᵢ) of less than 10 μM in α₇ nAChR receptor or α₄ nAChR receptor assays.

The compounds of the invention have the advantage that they may be less toxic, be more efficacious, be longer acting, have a broader range of activity, be more potent, produce fewer side effects, are more easily absorbed or have other useful pharmacological properties.

### General Experimental Procedures and Conditions

Commercial reagents were used without further purification. Mass spectra were recorded using either a Hewlett Packard 5988A or a MicroMass Quattro-1 Mass Spectrometer and are reported as m/z for the parent molecular ion. Room temperature refers to 20-25° C.

Microwave heating was achieved with a Personal Chemistry Smith Synthesizer or a Personal Chemistry Emrys Optimizer (monomodal, 2.45 GHz, 300 W max). Supercritical Fluid Chromatography (SFC) was performed as a means of purification for selected compounds and intermediates.

LC/MS HPLC method was generally performed with a Agilent Zorbax 5μ SB-C8 column 2.1 mm×5 cm. Solvents: A=H₂O with 0.05% TFA, B=10% H₂O, 90% Acetonitrile, 0.05% TFA. Gradient: (10-90% B over 3 min., 90% B hold through 4 min. 10% B at 5 min. and hold at 10% B until 6 min).

### Methods of Preparation

Methods which may be used for the synthesis of compounds of formula I, include the methods described below in the Schemes.

In the reaction schemes and text that follow D, E and G unless otherwise indicated, are as defined above for compounds of formula I and J, L and M are as defined herein.

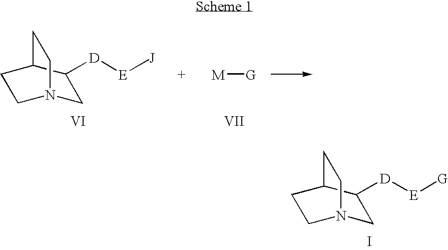

Compounds of formula I may be prepared from compounds of formula VI wherein J is a suitable leaving group by reaction with an appropriate organometallic compound of formula VII in the presence of an appropriate organometallic compound, for example, an arylboronic acid, in the presence of an organopalladium catalyst, for example, tetrakis(triphenylphosphine)palladium (0) or palladium (II) acetate and a base, for example cesium carbonate or potassium carbonate in an appropriate solvent, for example tetrahydrofuran, ethanol or toluene or mixtures thereof. The reaction can be conducted at a temperature between 20° C. and 120° C., preferably at or about reflux temperature of the solvent.

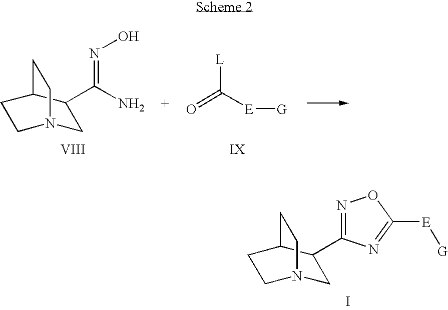

Compounds of formula I wherein D is a moiety of formula IV as shown in Scheme 2 may be prepared from compounds of formula VIII by reaction with a compound of formula IX, wherein L represents a suitable leaving group, using a suitable acylation procedure. Suitable leaving groups L include: OH, halogen, Oalkyl, Oaryl, OCOalkyl, OCOaryl, azide. A suitable acylation procedure involves treatment of a compound of formula VIII with a compound of formula IX at 0-120° C. in a suitable solvent. The presence of a base, or, when L is OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when L is OH include: carbodiimides, for example 1,3-dicyclohexylcarbodiimide or 1-(3-dimethylaminopropyl-3-ethylcarbodiimide hydrochloride; phosphonium reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

The compounds of formula VIII may be prepared by methods known to one skilled in the art (see, for example, J. Med. Chem., 33, 1128 (1990); J. Heterocyclic Chem., 28, 17 (1991); European J. Med. Chem., 39 (2004) 305-321; WO 9532965).

The compounds of formula IX are either commercially available, or may be prepared by methods known to one skilled in the art (see, for example, J. Heterocyclic Chem., 28, 17 (1991); European J. Med. Chem., 39 (2004) 305-321; WO 9532965).

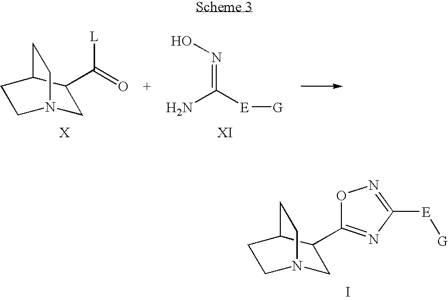

Compounds of formula I wherein D is a moiety of formula III as shown in Scheme 3 may be prepared from compounds of formula X by reaction with a compound of formula XI, wherein L represents a suitable leaving group, using a suitable acylation procedure. Suitable leaving groups L include: OH, halogen, Oalkyl, Oaryl, OCOalkyl, OCOaryl, azide. A suitable acylation procedure involves treatment of a compound of formula VII with a compound of formula XI at 0-120° C. in a suitable solvent. The presence of a base, or, when L is OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when L is OH include: carbodiimides, for example 1,3-dicyclohexylcarbodiimide or 1-(3-dimethylaminopropyl-3-ethylcarbodiimide hydrochloride; phosphonium reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

The compounds of formula X may be prepared by methods known to one skilled in the art (see, for example, J. Med. Chem., 33, 1128 (1990); J. Heterocyclic Chem., 28, 17 (1991)).

The compounds of formula XI are either commercially available, or may be prepared by methods known to one skilled in the art (see, for example, J. Heterocyclic Chem., 28, 17 (1991); European J. Med. Chem., 39 (2004) 305-321; WO 9532965).

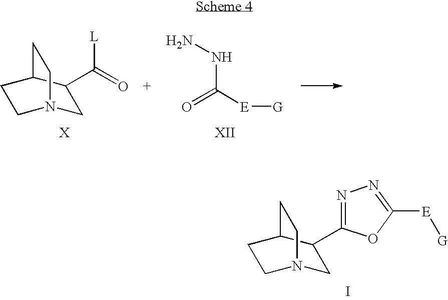

Compounds of formula I wherein D is a moiety of formula III as shown in Scheme 4 may be prepared from compounds of formula X by reaction with a compound of formula XII, wherein L represents a suitable leaving group, using a suitable acylation procedure. Suitable leaving groups L include: OH, halogen, Oalkyl, Oaryl, OCOalkyl, OCOaryl, azide. A suitable acylation procedure involves treatment of a compound of formula X with a compound of formula XII at 0-120° C. in a suitable solvent. The presence of a base, or, when L is OH, a coupling agent, may also be necessary for the reaction to occur. Suitable bases for the reaction include: 4-(N,N-dimethylamino)pyridine, pyridine, triethylamine, N,N-diisopropylethylamine. The preferred base is N,N-diisopropylethylamine. Suitable coupling agents when L is OH include: carbodiimides, for example 1,3-dicyclohexylcarbodiimide or 1-(3-dimethylaminopropyl-3-ethylcarbodiimide hydrochloride; phosphonium reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate. The preferred coupling agent is O-benzotriazol-1-yl-N,N,N′,N′-tetramethyluronium tetrafluoroborate. Suitable solvents for the reaction include N,N-dimethylformamide, dimethylsulfoxide, tetrahydrofuran, or chloroform. The preferred solvent is N,N-dimethylformamide. The reaction is preferably performed at a temperature of 0-50° C., and most preferably at a temperature of 20-30° C.

The compounds of formula XII are either commercially available, or may be prepared by methods known to one skilled in the art (see, for example, J. Heterocyclic Chem., 28, 17 (1991); JOC, 1999, 64, 6989).

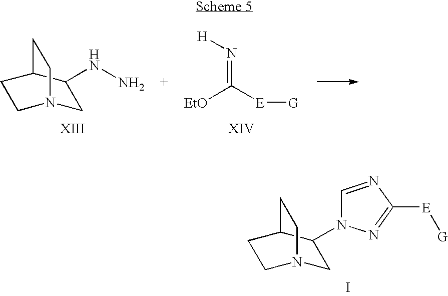

Compounds of formula I wherein D is a moiety of formula V as shown in Scheme 5 may be prepared from compounds of formula XIII by reaction with a compound of formula XIV by methods known to those skilled in the art (see, for example, J. Med. Chem., 1992, 35, 2392-2406).

The compounds of formula X may be prepared by methods known to one skilled in the art (see, for example, J. Med. Chem., 35, 1280 (1992). The compounds of formula XIV may be prepared by methods known to one skilled in the art (see, for example, Chem. Rev.; 61, 179 (1961).

It will be appreciated by those skilled in the art that aromatic substituents in the compounds of the invention, or in intermediates used in the synthesis of compounds of the invention, may be introduced by employing aromatic substitution reactions, functional group transformations to modify existing substituents, or a combination thereof. Such reactions may be effected either prior to or immediately following the processes mentioned above. The reagents and reaction conditions for such procedures are known in the art. Specific examples of procedures which may be employed include, but are not limited to, electrophilic functionalisation of an aromatic ring, for example by nitration, halogenation, or acylation; transformation of a nitro group to an amino group, for example by reduction, such as by catalytic hydrogenation; acylation, alkylation, sulfonylation of an amino or hydroxyl group; replacement of an amino group by another functional group by conversion to an intermediate diazonium salt followed by nucleophilic or free radical substitution of the diazonium salt; or replacement of a halogen by another functional group, for example by nucleophilic or organometallically-catalysed substitution reactions.

Where necessary, hydroxy, amino, or other reactive groups may be protected using protecting groups by standard techniques.

The above-described reactions, unless otherwise noted, are usually conducted at a pressure of about one to about three atmospheres, preferably at ambient pressure (about one atmosphere).

Unless otherwise stated, the above-described reactions are conducted under an inert atmosphere, preferably under a nitrogen atmosphere.

The compounds of the invention and intermediates may be isolated from their reaction mixtures by standard techniques.

Acid addition salts of the compounds of formula I which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

When compounds of formula I exist in tautomeric or enantiomeric forms, all of such forms are included within the scope of the invention. Optical isomers of compounds of the invention may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemisation.

### EXAMPLES

### Example 1

### 3-(5-(5-phenylthiophen-2-yl)-1,3,4-oxadiazol-2- quinuclidine

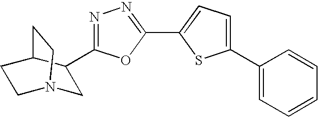

A stirred mixture of 3-(5-(5-bromothiophen-2-yl)-1,3,4-oxadiazol-2-yl)quinuclidine (40 mg, 0.12 mmol), phenylboronic acid (80 mg, 0.65 mmol), potassium carbonate (400 mg, 2.9 mmol), and tetrakis(triphenylphosphine)palladium(0) (10 mg, 0.008 mmol) in a solvent mixture of THF, EtOH, and water (1.5 mL each) was heated at reflux temperature for 4 h. The cooled reaction mixture was concentrated to near dryness and triturated with chloroform (3×), dried over sodium sulfate and concentrated. The resulting product was purified by flash chromatography on silica gel eluting with ammoniated chloroform to give the title compound (30 mg) as a white solid. MS (ES+) 338 (MH+). 1H NMR (300.132 MHz, CDCl3) 7.68 (d, J=3.9 Hz, 1H), 7.67-7.62 (m, 2H), 7.46-7.36 (m, 3H), 7.34 (d, J=3.9 Hz, 1H), 3.49 (dd, J=13.6, 6.2 Hz, 1H), 3.35 (dd, J=10.0, 2.2 Hz, 1H), 3.24-3.17 (m, 1H), 3.11-2.82 (m, 3H), 2.28 (q, J=3.0 Hz, 1H), 1.79-1.71 (m, 4H), 1.54-1.41 (m, 1H).

The starting material, 3-(5-(5-bromothiophen-2-yl)-1,3,4-oxadiazol-2-yl)quinuclidine was prepared as follows:

a. To a vigorously stirred mixture of quinuclidine-3-carboxylic acid hydrochloride (190 mg, 1.0 mmol), 2-chloro-1,3-dimethylimidazolinium chloride (0.7 g, 4.1 mmol), and 5-bromo-2-thiophenecarboxylic acid hydrazide (230 mg, 1.0 mmol) in dry acetonitrile (6 mL) at ambient temperature was added dropwise triethylamine (1.3 mL). The resulting orange-colored mixture was stirred for 5 minutes, heated at reflux temperature for 1 hour, and then cooled. The resulting residue obtained after removing the volatiles by rotary evaporation was purified by flash chromatography on silica gel eluting with a ammoniated chloroform to 6% methanol/chloroform gradient to give 3-(5-(5-bromothiophen-2-yl)-1,3,4-oxadiazol-2-yl)quinuclidine (40 mg) as an off-white solid. MS (ES+) 340/2 (MH+). NMR (CHCl₃)

b. The quinuclidine-3-carboxylic acid hydrochloride was prepared by methods described in J. Med. Chem., 34, 2726 (1991).

### Example 2

### 3-(3-(5-phenylthiophen-2-yl)-1,2,4-oxadiazol-5-yl)quinuclidine

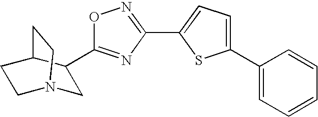

Using a procedure similar to that described in Example 1 but substituting 3-(5-(5-bromothiophen-2-yl)-1,2,4-oxadiazol-2-yl)quinuclidine for 3-(5-(5-bromothiophen-2-yl)-1,3,4-oxadiazol-2-yl)quinuclidine gave the title compound as a white solid. MS (ES+) 338 (MH+). ¹H NMR (300.132 MHz, CDCl₃) 7.77 (d, J=3.9 Hz, 1H), 7.70-7.62 (m, 2H), 7.46-7.36 (m, 3H), 7.35 (d, J=3.9 Hz, 1H), 3.58-3.47 (m, 1H), 3.44-3.32 (m, 1H), 3.31-3.22 (m, 1H), 3.14-2.86 (m, 3H), 2.34 (q, J=2.8 Hz, 1H), 1.84-1.41 (m, 5H).

The starting material, 3-(5-(5-bromothiophen-2-yl)-1,2,4-oxadiazol-2-yl)quinuclidine, was prepared as follows:

a. To a stirred mixture of 5-bromo-N-hydroxy-thiophene-2-carboxamidine (300 mg, 1.36 mmol) and triethylamine (0.25 mL, 1.8 mmol) in anhydrous chloroform (3 mL) at ambient temperature was added dropwise a solution of quinuclidine-3-carbonyl chloride (200 mg, 1.16 mmol) in dry chloroform (1.5 mL). The mixture was stirred for 24 hours, the volatiles removed and replaced with dry acetonitrile (4 mL). The resulting solution was subjected to microwave irradiation at 120° C. for 30 min. The cooled mixture was concentrated and the residue purified by flash chromatography on silica gel eluting with a ammoniated chloroform to 4% MeOH/chloroform gradient to give the title compound (100 mg) as a solid. MS (ES+) 340/2 (MH+). NMR (CHCl₃).

b. The 5-bromo-N-hydroxy-thiophene-2-carboxamidine used above was prepared by methods described in J. Heterocyclic Chem., 28, 17 (1991).

c. The quinuclidine-3-carbonyl chloride used above was prepared by methods described in J. Med. Chem., 33, 1128 (1990).

### Example 3

### 3-[3-(5-Pyridin-3-yl-furan-2-yl)-[1,2,4]oxadiazol-5-yl]quinuclidine

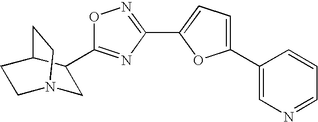

Using a procedure similar to that described in Example 2 but substituting 5-bromo-N-hydroxy-furan-2-carboxamidine for the 5-bromo-N-hydroxy-thiophene-2-carboxamidine in part (a), and substituting 3-(4,4,5,5-tetramethyl-1,3,2-dioxaborolan-2-yl)-pyridine for the phenylboronic acid as in Example 1 gave the title compound as a white solid. MS (ES+) 323 (H+). ¹H NMR (300.132 MHz, CDCl₃) 9.03 (d, J=1.6 Hz, 1H), 8.57 (dd, J=4.8, 1.6 Hz, 1H), 8.10 (dt, J=8.0, 1.9 Hz, 1H), 7.36 (dd, J=8.0, 4.8 Hz, 1H), 7.25 (d, J=3.6 Hz, 1H), 6.91 (d, J=3.6 Hz, 1H), 3.48 (dd, J=13.5, 6.2 Hz, 1H), 3.42-3.31 (m, 1H), 3.31-3.21 (m, 1H), 3.12-2.82 (m, 3H), 2.32 (q, J=2.9 Hz, 1H), 1.81-1.42 (m, 5H).

a. The 5-bromo-N-hydroxy-furan-2-carboxamidine used above was prepared by methods described in J. Heterocyclic Chem., 28, 17 (1991).

### Example 4

### 3-[3-(5-Pyridin-3-yl-thiophen-2-yl)-[1,2,4]oxadiazol-5-yl]quinuclidine

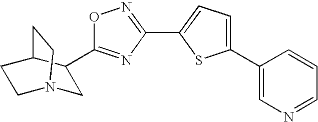

Using a procedure similar to that described in Example 2 and substituting 3-(4,4,5,5-tetramethyl-1,3,2-dioxaborolan-2-yl)pyridine for the phenylboronic acid as in Example 1 gave the title compound as a white solid. MS (ES+) 339 (MH+). ¹H NMR (300.132 MHz, CDCl₃) 8.94 (d, J=1.9 Hz, 1H), 8.58 (dd, J=4.8, 1.4 Hz, 1H), 7.91 (dt, J=8.0, 0.8 Hz, 1H), 7.81 (d, J=3.8 Hz, 1H), 7.40 (d, J=3.9 Hz, 1H), 7.35 (dd, J=8.0, 4.8 Hz, 1H), 3.47 (dd, J=13.5, 6.3 Hz, 1H), 3.42-3.30 (m, 1H), 3.29-3.21 (m, 1H), 3.12-2.83 (m, 3H), 2.31 (q, J=3.0 Hz, 1H), 1.81-1.40 (m, 5H).

### Example 5

### 3-[5-(5-Phenyl-furan-2-yl)-[1,3,4]oxadiazol-2-yl]quinuclidine

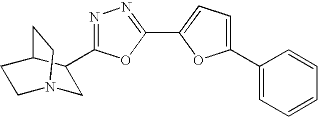

Using a procedure similar to that described in Example 1 but substituting 5-bromo-2-furancarboxylic acid hydrazide for 5-bromo-2-thiophenecarboxylic acid hydrazide gave the title compound as a white solid. MS (ES+) 322 (MH+). ¹H NMR (300.132 MHz, CDCl₃) 7.80 (d, J=7.1 Hz, 2H), 7.46-7.33 (m, 3H), 7.21 (d, J=3.6 Hz, 1H), 6.81 (d, J=3.6 Hz, 1H), 3.59-3.48 (m, 1H), 3.43-3.30 (m, 1H), 3.29-3.19 (m, 1H), 3.13-2.84 (m, 3H), 2.31 (q, J=2.9 Hz, 1H), 1.83-1.41 (m, 5H).

### Example 6

### 3-[5-(5-Pyridin-3-yl-furan-2-yl)-[1,3,4]oxadiazol-2-yl]quinuclidine

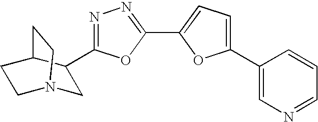

Using a procedure similar to that described in Example 1 but substituting 5-bromo-2-furancarboxylic acid hydrazide for 5-bromo-2-thiophenecarboxylic acid hydrazide and substituting 3-(4,4,5,5-tetramethyl-1,3,2-dioxaborolan-2-yl)-pyridine for phenylboronic acid gave the title compound as a white solid. MS (ES+) 323 (MH+). ¹H NMR (300.132 MHz, CDCl₃) 9.03. (d, J=1.6 Hz, 1H), 8.59 (dd, J=4.8, 1.6 Hz, 1H), 8.09 (dt, J=8.0, 1.9 Hz, 1H), 7.38 (dd, J=8.0, 4.8 Hz, 1H), 7.24 (d, J=3.8 Hz, 1H), 6.92 (d, J=3.6 Hz, 1H), 3.54 (dd, J=13.3, 6.1 Hz, 1H), 3.44-3.31 (m, 1H), 3.30-3.20 (m, 1H), 3.14-2.84 (m, 3H), 2.32 (q, J=3.0 Hz, 1H), 1.83-1.42 (m, 5H).

### Example 7

### 3-[5-(5-Pyridin-3-yl-thiophen-2-yl)-[1,2,4]oxadiazol-3-yl]quinuclidine

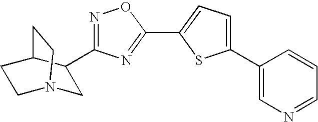

Using a procedure similar to that described in Example 1 but substituting 3-[5-(5-bromo-thiophen-2-yl)-[1,2,4]oxadiazol-3-yl]quinuclidine for 3-[5-(5-bromo-thiophen-2-yl)-[1,3,4]oxadiazol-2-yl]quinuclidine and substituting 3-(4,4,5,5-betramethyl-1,3,2-dioxaborolan-2-yl)-pyridine for the phenylboronic acid gave the title compound. MS (ES+) 339 (MH+). 1H NMR (300.132 MHz, CDCl₃) 8.94 (d, J=2.3 Hz, 1H), 8.62 (dd, J=4.8, 1.5 Hz, 1H), 7.92 (ddd, J=8.0, 2.3, 1.6 Hz, 1H), 7.88 (d, J=4.0 Hz, 1H), 7.43 (d, J=3.9 Hz, 1H), 7.38 (ddd, J=8.0, 4.8, 0.8 Hz, 1H), 3.45-3.26 (m, 2H), 3.18-2.82 (m, 4H), 2.25 (q, J=2.9 Hz, 1H), 1.84-1.37 (m, 5H).

The starting material, 3-[5-(5-bromo-thiophen-2-yl)-[1,2,4]oxadiazol-3-yl]quinuclidine, was prepared as follows:

a. A stirred mixture of N-hydroxy-quinuclidine-3-carboxamidine hydrochloride (480 mg, 2.3 mmol) and 5-bromo-thiophene-2-carbonyl chloride (570 mg, 2.5 mmol) in dry acetonitrile (3 mL) was subjected to microwave irradiation at 120° C. for 30 minutes. The cooled mixture was treated with triethylamine (0.2 mL) and dry DMF (0.5 mL) and subjected to microwave irradiation at 120° C. for 20 minutes. The volatiles were removed and the resulting product was purified by flash chromatography on silica gel eluting with a ammoniated chloroform to 4% MeOH/chloroform gradient to give 3-[5-(5-bromo-thiophen-2-yl)-[1,2,4]oxadiazol-3-yl]quinuclidine (250 mg) as a syrup which crystallized on standing. MS (ES+) 340/2 (MH+).

b. The N-hydroxyquinuclidine-3-carboxamidine hydrochloride used above was prepared by methods described in J. Med. Chem., 34, 2726 (1991).

### Example 8

### 3-[5-(5-Pyridin-3-yl-furan-2-yl)-[1 2,4]oxadiazol-3-yl]quinuclidine

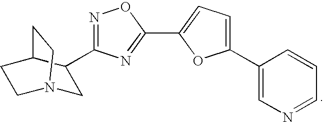

Using a procedure similar to that described in Example 7 but substituting 5-bromo-furan-2-carbonyl chloride for 5-bromo-thiophene-2-carbonyl chloride in part (a) resulted in the title compound. MS (ES+) 323 (MH+). ¹H NMR (300.132 MHz, CDCl₃) 9.06 (d, J=2.2 Hz, 1H), 8.61 (dd, J=4.8, 1.6 Hz, 1H), 8.13 (dt, J=8.0, 2.0 Hz, 1H), 7.41 (d, J=3.7 Hz, 1H), 7.38 (dd, J=4.9, 0.8 Hz, 1H), 6.95 (d, J=3.7 Hz, 1H), 3.47-3.27 (m, 2H), 3.16 (ddt, J=9.3, 7.2, 2.1 Hz, 1H), 3.09-2.79 (m, 3H), 2.27 (q, J=2.9 Hz, 1H), 1.84-1.64 (m, 4H), 1.53-1.36 (m, 1H).

### Example 9

### 3-{5-[Quinuclidin-3-yl)-[1,2,4]oxadiazol-5-yl]-furan-2-yl}-N,N-dimethyl-benzamide

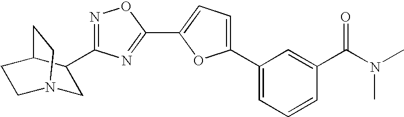

Using a procedure similar to that described in Example 7 but substituting 5-bromo-furan-2-carbonyl chloride for 5-bromo-thiophene-2-carbonyl chloride in part (a) and using a procedure similar to that described in Example 1 but substituting 3-(N,N-dimethylcarbonyl)phenylboronic acid for phenylboronic acid resulted in the title compound. MS (ES+) 393 (MH+). ¹H NMR (300.132 MHz, CDCl₃) 7.88-7.85 (m, 2H), 7.49 (t, J=8.0 Hz, 1H), 7.42-7.38 (m, 1H), 7.38 (d, J=3.7 Hz, 1H), 6.88 (d, J=3.7 Hz, 1H), 3.49-3.30 (m, 2H), 3.20-2.84 (m, 11H), 2.30-2.27 (m, 1H), 1.85-1.39 (m, 4H).

### Example 10

### 3-{5-[5-(Quinuclidin-3-yl)-[1,3,4]oxadiazol-2-yl]-furan-2-yl}-N,N-dimethyl-benzamide

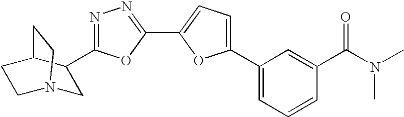

Using a procedure similar to that described in Example 1 but substituting 5-bromo-2-furancarboxylic acid hydrazide for 5-bromo-2-thiophenecarboxylic acid hydrazide in part (a) and substituting 3-(N,N-dimethylcarbonyl)-phenylboronic acid for phenylboronic acid gave the title compound. MS (ES+) 393 (MH+). ¹H NMR (300.132 MHz, CDCl₃) 7.86-7.82 (m, 2H), 7.48 (t, J=7.7 Hz, 1H), 7.38 (dd, J=6.3, 1.3 Hz, 1H), 7.22 (d, J=3.7 Hz, 1H), 6.85 (d, J=3.6 Hz, 1H), 3.57-3.49 (m, 1H), 3.41-3.33 (m, 1H), 3.26-2.86 (m, 11H), 2.33-2.30 (m, 1H), 1.83-1.43 (m, 4H).

### Example 11

### 3-[5-(5-Pyridin-3-yl-oxazol-2-yl)-[1,2,4]oxadiazol-3-yl]quinuclidine

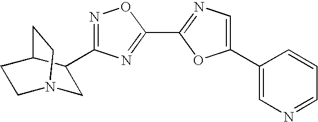

A mixture of N-hydroxyquinuclidine-3-carboxamidine hydrochloride (205 mg, 1.0 mmol), lithium 5-pyridin-3-yl-oxazole-2-carboxylate (200 mg, 1.0 mmol), N,N-diisopropylethylamine (0.8 mL, 4.6 mmol), O-benzotriazol-1-yl-N,N,N′,N′-tetramethyluronium tetrafluoroborate (420 mg, 1.3 mmol), 1-hydroxybenzotriazole hydrate (180 mg, 1.3 mmol), and powdered 4A molecular sieves (700 mg) in dry DMF (12 mL) was stirred vigorously at room temperature for 2 days, then subjected to microwave irradiation at 100° C. for 15 minutes. The volatiles were removed in vacuo and the product residue purified by flash chromatography on silica gel eluting with a ammoniated chloroform to 3% MeOH/chloroform gradient to give the title compound (85 mg) as a white solid. MS (ES+) 324 (MH+). ¹H NMR (300.132 MHz, CDCl₃) 9.07 (d, J=1.8 Hz, 1H), 8.68 (dd, J=4.9, 1.5 Hz, 1H), 8.10 (dt, J=8.0, 1.9 Hz, 1H), 7.77 (s, 1H), 7.44 (dd, J=7.7, 5.1 Hz, 1H), 3.48-3.32 (m, 2H), 3.25-3.18 (m, 1H), 3.10-2.81 (m, 4H), 2.30 (q, J=2.9 Hz, 1H), 1.83-1.40 (m, 4H).

The lithium 5-pyridin-3-yl-oxazole-2-carboxylate used above was prepared as follows:

### a. (2-Oxo-2-pyridin-3-yl-ethyl)-carbamic acid tert-butyl ester

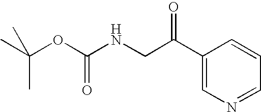

To a solution of 3-bromopyridine (1.21 mL, 12.6 mmol) in 15 mL of dry THF was added isopropylmagnesium chloride (2M in THF, 6.3 mL, 12.6 mmol) at room temperature under N₂. After 45 min., in a separate flask, isopropylmagnesium chloride (4.9 mL, 9.8 mmol) was added to a cooled (−15 to −10° C.) slurry of N-(tert-butoxycarbonyl)glycine N′-methoxy-N′-methylamide (2.18 g, 10.0 mmol) in 15 mL of dry THF under N₂. After the Br—Mg exchange reaction had stirred for a total of 1 hr, the resulting mixture was added to the Weinreb amide anion solution. After the entire contents had been added, the reaction mixture was allowed to warm to room temperature and stirred overnight. The mixture was then partitioned between EtOAc and water. The layers were separated and the aqueous layer was extracted with EtOAc The organic extracts were combined, dried over MgSO₄, filtered and concentrated in vacuo. The residue was chromatographed on silica gel (100% Hexane to 25% EtOAc/Hexane gradient) to give 1.57 g of a white solid as desired product (66%). ¹H-NMR: 300 MHz, CDCl₃ δ 9.17 (m, 1H); 8.82 (m, 1H); 8.23 (m, 1H); 7.44 (m, 1H); 5.45 (br s, 1H); 4.66 (d, 2H); 1.48 (s, 9H).

### b. 2-Amino-1-pyridin-3-yl-ethanone dihydrochloride

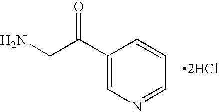

To a solution of (2-oxo-2-pyridin-3-yl-ethyl)-carbamic acid tert-butyl ester in MeOH (7 mL) was added 5-6N HCl in 2-propanol (7 mL). The mixture was heated at 50° C. for 2 hr, then concentrated in vacuo and dried on high vacuum. The quantitative yield of off-white solid was used without further purification.

### c. N-(2-Oxo-2-pyridin-3-yl-ethyl)-oxalamic acid ethyl ester

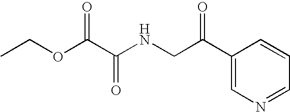

To a cooled (ice bath) mixture of 2-amino-1-pyridin-3-yl-ethanone dihydrochloride (913 mg, 4.37 mmol) and ethyl chlorooxoacetate (0.54 mL, 4.8 mmol) in 15 mL of CH₂Cl₂ was added triethylamine (1.9 mL, 13.6 mmol). The resulting reaction mixture was stirred at room temperature overnight. The mixture was then partitioned between CH₂Cl₂ and water. The layers were separated and the aqueous layer was extracted with CH₂Cl₂. The organic extracts were combined, dried over MgSO₄, filtered and concentrated in vacuo. The residue was purified by column chromatography on silica gel (20% EtOAc in Hexane to 80% EtOAc in Hexane gradient). LC/MS (APcI): (M+H)⁺=237.1

### d. 5-Pyridin-3-yl-oxazole-2-carboxylic acid ethyl ester

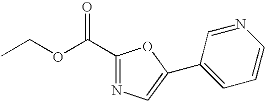

To a cooled (ice bath) mixture of N-(2-oxo-2-pyridin-3-yl-ethyl)-oxalamic acid ethyl ester (750 mg, 3.18 mmol), triphenylphosphine (1.89 g, 7.21 mmol), and hexachloroethane (1.55 g, 6.55 mmol) in 30 mL CH₂Cl₂ was added triethylamine (1.67 mL, 11.96 mmol). The reaction mixture was stirred for 1 hr, then chromatographed on silica gel (EtOAc/Hexane gradient). 700 mg of an off-white solid were collected corresponding to desired oxazole and containing a small amount of triphenylphosphine oxide. ¹H-NMR: 300 MHz, CDCl₃ δ 9.02 (s, 1H); 8.65 (m, 1H); 8.06 (m, 1H); 7.62 (s, 1H); 7.42 (m, 1H); 4.51 (quart, 2H); 1.47 (t, 3H). LC/MS (APcI): (M+H)⁺=219.1.

### e. Lithium 5-pyridin-3-yl-oxazole-2-carboxylate

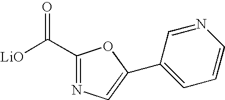

A solution of LiOH.H₂O (133 mg, 3.18 mmol) in 7 mL of water was added to a stirring solution of 5-pyridin-3-yl-oxazole-2-carboxylic acid ethyl ester (700 mg, 3.18 mmol) in 7 mL of THF. Methanol (1 mL) was added and the mixture was stirred overnight at room temp. The reaction mixture was then concentrated in vacuo and the resulting pale yellow solid was triturated with acetone. After removal of acetone and drying under high vacuum, 530 mg of an off-white solid was obtained.

The N-hydroxyquinuclidine-3-carboxamidine hydrochloride used above was prepared by methods described in J. Med. Chem., 34, 2726 (1991).

### Example 12

### 3-[5-(5-Pyridin-4-yl-oxazol-2-yl)-[1,2,4]oxadiazol-3-yl]quinuclidine

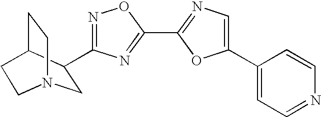

Using a procedure similar to that described in Example 11 but substituting lithium 5-pyridin-4-yl-oxazole-2-carboxylate for the lithium 5-pyridin-3-yl-oxazole-2-carboxylate gave the title compound. MS (ES+) 324 (MH+). ¹H NMR (300.132 MHz, CDCl₃) δ 8.77 (dd, J=4.6, 1.5 Hz, 2H), 7.84 (s, 1H), 7.66 (dd, J=4.5, 1.6 Hz, 2H), 3.51-3.30 (m, 2H), 3.26-3.21 (m, 1H), 3.13-2.81 (m, 4H), 2.34-2.25 (m, 1H), 1.84-1.40 (m, 4H).

The lithium 5-pyridin-4-yl-oxazole-2-carboxylate used above was prepared as follows:

### a. (2-Oxo-2-pyridin-4-yl-ethyl)-carbamic acid tert-butyl ester

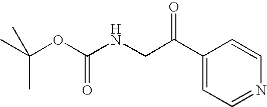

4-Bromopyridine hydrochloride (2.45 g, 12.6 mmol) was treated with 65 mL of 5% aqueous Na₂CO₃ and extracted twice with 30 mL Et₂O. The ethereal extracts were dried over MgSO₄, filtered and the solvent removed in vacuo. The residue was immediately dissolved in dry THF and isopropylmagnesium chloride (2 M in THF, 6.3 mL, 12.6 mmol) was added at room temp under N₂. After 45 min., in a separate flask, isopropylmagnesium chloride (4.9 mL, 9.8 mmol) was added to a cooled (−15 to −10° C.) slurry of N-(tert-butoxycarbonyl)glycine N′-methoxy-N′-methylamide (2.18 g, 10.0 mmol) in 15 mL of dry THF under N₂. After the Br—Mg exchange reaction had stirred for a total of 1 hr, the resulting mixture was added to the Weinreb amide anion solution. After the entire contents had been added, the reaction mixture was allowed to warm to room temperature and stirred overnight. The mixture was then partitioned between EtOAc and water. The layers were separated and the aqueous layer was extracted with EtOAc. The organic extracts were combined, dried over MgSO₄, filtered and concentrated in vacuo. The residue was chromatographed on silica gel (100% Hexane to 30% EtOAc/Hexane gradient) to give 1.2 g of an amber solid as desired product. ¹H-NMR: 300 MHz, CDCl₃ δ 8.67 (d, 1H); 8.04 (d, 1H); 7.85 (m, 1H); 7.5 (m, 1H); 5.36 (br s, 1H); 4.88 (d, 2H); 1.48 (s, 9H).

### b. 2-Amino-1-pyridin-4-yl-ethanone dihydrochloride

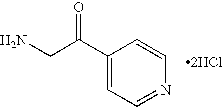

To a solution of (2-oxo-2-pyridin-4-yl-ethyl)-carbamic acid tert-butyl ester in MeOH (7 mL) was added 5-6 N HCl in 2-propanol (7 mL). The mixture was heated at 50° C. for 2 hr, then concentrated in vacuo and dried on high vacuum. The quantitative yield of off-white solid was carried on without further purification.

### c. 5-Pyridin-4-yl-oxazole-2-carboxylic acid ethyl ester

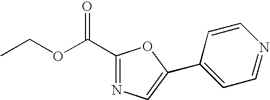

To a cooled (ice bath) mixture of 2-amino-1-pyridin-3-yl-ethanone dihydrochloride (5.08 mmol) and ethyl chlorooxoacetate (0.62 mL, 5.5 mmol) in 20 mL of CH₂Cl₂ was added triethylamine (2.26 mL, 16.25 mmol). The resulting reaction mixture was stirred at room temperature overnight. The mixture was then partitioned between CH₂Cl₂ and water. The layers were separated and the aqueous layer was extracted with CH₂Cl₂. The organic extracts were combined, dried over MgSO₄, filtered and concentrated in vacuo. LC/MS (APcI): (M+H)⁺=219.1 corresponding to cyclized oxazole was observed as the major component in the product mixture. A smaller peak corresponding to the uncyclized amide ((M+H)⁺=237.1) was also observed. The mixture was chromatographed on silica gel (100% Hexane to 35% EtOAc in Hexane gradient) to give 142 mg of oxazole product (13%).

### d. Lithium 5-pyridin-4-yl-oxazole-2-carboxylate

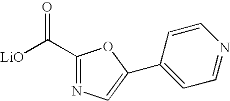

A solution of LiOH.H₂O (30 mg, 0.17 mmol) in 3 mL of water was added to a stirring solution of 5-pyridin-4-yl-oxazole-2-carboxylic acid ethyl ester (140 mg, 0.64 mmol) in 3 mL of THF. 0.5 mL of MeOH was added and the mixture was stirred overnight at room temp. The reaction mixture was then concentrated in vacuo and the resulting pale yellow solid was triturated with acetone. After removal of acetone and drying under high vacuum, quantitative yield of an off-white solid was obtained. ¹H-NMR: 300 MHz, dmso-d₆ δ 8.62 (d, 2H); 7.83 (s, 1H); 7.64 (d, 2H). LC/MS (APcI): (M+)⁺=191.1.

### Example 13

### 3-(3-(5-(pyridin-3-yl)thiophen-2-yl)-1H-1,2,4-triazol-1-yl)quinuclidine

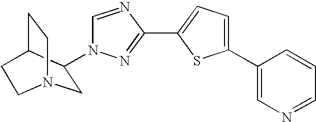

A mixture of 3-(3-(5-bromothiophen-2-yl)-1H-1,2,4-triazol-1-yl)quinuclidine (120 mg), pyridine-3-boronic acid (52 mg), cesium carbonate (234 mg) and bistriphenylphosphine palladium(II) chloride (13 mg) in 1,2-dimethoxyethane/water/ethanol (7:3:2, 2.5 mL) was subjected to microwave irradiation at 150° C. for 10 minutes. After cooling to room temperature, the mixture was partitioned between chloroform (2×25 mL) and saturated aqueous potassium carbonate (2×25 mL). The organic extracts were combined, dried over potassium carbonate, filtered and evaporated to leave a yellow oil (110 mg). The product was purified by flash chromatography on basic alumina eluting with a chloroform to 1% MeOH/chloroform gradient followed by preparative supercritical fluid chromatography (Chiralpak AD, 17% MeOH, 0.5% DMEA in CO₂) to afford the title compound, which was isolated as the dihydrochloride salt (10 mg). MS (AP+) 338 (MH+). ¹H NMR (300.132 MHz, CDCl₃) δ 8.93 (s, 1H), 8.53 (d, J=3.8 Hz, 1H), 8.14 (s, 1H), 7.90 (d, J=7.9 Hz, 1H), 7.68 (d, J=3.8 Hz, 1H), 7.36 (d, J=3.8 Hz, 2H), 7.32 (dd, J=8.0, 4.9 Hz, 2H), 4.45 (s, 1H), 3.54-3.40 (m, 2H), 3.20-3.11 (m, 1H), 3.01-2.84 (m, 4H), 2.24 (q, J=2.9 Hz, 1H), 1.88-1.70 (m, 4H), 1.53-1.43 (m, 1H).

The starting material, 5-bromothiophene-2-carboximidic acid ethyl ester hydrochloride, was prepared as follows:

### a. 5-Bromothiophene-2-carboximidic acid ethyl ester hydrochloride

A solution of 2-bromo-5-cyanothiophene (1.05 g) in diethyl ether (20 mL) and ethanol (1 mL) was treated with hydrogen chloride (g) for 30 min. The resulting mixture was stirred at room temperature for 72 hr to afford a white precipitate. The product was isolated by filtration, washed with cold diethyl ether (2×5 mL) and dried in vacuo to afford the title compound as a white solid (1.20 g). ¹H NMR (300.132 MHz, DMSO) δ 8.52 (s, 1H), 7.56 (s, 1H), 4.61 (q, J=7.0 Hz, 2H), 1.42 (t, J=7.0 Hz, 3H). 13C NMR (75.477 MHz, DMSO) δ 164.21, 137.54, 133.31, 130.23, 124.13, 70.17, 13.85.

### b. 3-(3-(5-Bromothiophen-2-yl)-1H-1,2,4-triazol-1-yl)quinuclidine

A solution of 1-(quinuclidin-3-yl)hydrazine dihydrochloride (213 mg) and 5-bromothiophene-2-carboximidic acid ethyl ester hydrochloride (284 mg) and triethylamine (313 mg) in methanol (5 mL) was stirred at room temperature for 22 hr. The solvent was then evaporated and the residue was suspended in triethyl orthoformate (10 mL) and pyridine (2 mL). The resulting mixture was heated to reflux for 16 hr, then was cooled to room temperature and concentrated. The residue was partitioned between ethyl acetate (3×50 mL) and saturated aqueous potassium carbonate (2×20 mL). The organic extracts were combined, dried over potassium carbonate, filtered and evaporated to leave an amber oil (295 mg). The product was purified by flash chromatography on basic alumina eluting with a chloroform to 2% MeOH/chloroform gradient to afford the title compound as a clear oil (120 mg). MS (AP+) 339/341 (MH+). ¹H NMR (300.132 MHz, CDCl₃) δ 8.09 (s, 1H), 7.41 (d, J=3.8 Hz, 1H), 7.04 (d, J=3.9 Hz, 1H), 4.45-4.38 (m, 1H), 3.45-3.42 (m, 2H), 3.18-3.06 (m, 1H), 2.95-2.87 (m, 4H), 2.23-2.20 (m, 1H), 1.82-1.73 (m, 4H), 1.58 (s, 3H).

### c. The 1-(quinuclidin-3-yl)hydrazine dihydrochloride used above was prepared by methods described in J. Med. Chem., 35, 1280 (1992).

### Example 14

### 3-[3-(5-Phenyl-thiophen-2-yl)-[1,2,4]triazol-1-yl]quinuclidine

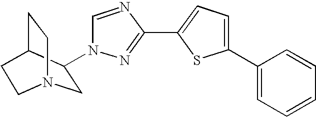

Using a procedure similar to that described in Example 1 but substituting 3-[3-(5-bromo-thiophen-2-yl)-[1,2,4]triazol-1-yl]quinuclidine for 3-[5-(5-bromo-thiophen-2-yl)-[1,3,4]oxadiazol-2-yl]quinuclidine gave the title compound (80 mg) as an off-white solid. MS (ES+) 337 (MH+). ¹H NMR (300.132 MHz, CDCl₃) 8.17 (s, 1H), 7.67-7.63 (m, 3H), 7.39 (t, J=7.5 Hz, 2H), 7.34-7.28 (m, 2H), 4.46-4.41 (m, 1H), 3.54-3.39 (m, 2H), 3.20-3.10 (m, 1H), 3.01-2.84 (m, 3H), 2.27-2.23 (m, 1H), 1.90-1.40 (m, 4H).

The starting material, 3-[3-(5-bromo-thiophen-2-yl)-[1,2,4]triazol-1-yl]quinuclidine, was prepared as follows:

a. To a stirred mixture of 1-(quinuclidin-3-yl)hydrazine dihydrochloride (105 mg, 0.5 mmol) and triethylamine (0.3 mL, 2.0 mmol) in anhydrous ethanol (3 mL) was added in one portion 5-bromo-thiophene-2-carboximidic acid ethyl ester hydrochloride (135 mg, 0.5 mmol). The resulting yellow-green solution was stirred at room temperature for 12 hours, treated with triethyl orthoformate (0.5 mL, 3.0 mmol), and subjected to microwave irradiation at 120° C. for 20 minutes. The volatiles were removed in vacuo and the product residue was purified by flash chromatography on silica gel eluting with a ammoniated chloroform to 2% MeOH/chloroform gradient to give 3-[3-(5-bromo-thiophen-2-yl)-[1,2,4]triazol-1-yl]quinuclidine (125 mg) as a white solid. MS (ES+) 339/41 (MH+).
b. The 1-(quinuclidin-3-yl)-hydrazine dihydrochloride used above was prepared by methods described in J. Med. Chem., 35, 1280 (1992).

1. A compound according to formula I: 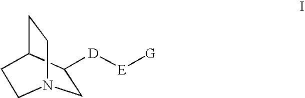
wherein:
D is selected from moieties according to formula II, III, IV or V 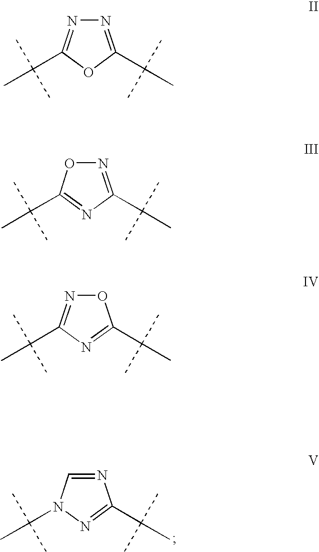
E and G are independently selected from a 5- or 6-membered aromatic or heteroaromatic ring having 0, 1 or 2 nitrogen atoms, 0 or 1 oxygen atoms, and 0 or 1 sulfur atoms, or selected from an 8-, 9- or 10-membered fused aromatic or heteroaromatic ring system having 0, 1, 2 or 3 nitrogen atoms, 0 or 1 oxygen atoms, and 0 or 1 sulfur atoms;
wherein E and G are unsubstituted or independently have 1, 2 or 3 substituents selected from —C₁-C₆alkyl, —C₂-C₆alkenyl, —C₂-C₆alkynyl, halogen, —CN, —NO₂, —CF₃, —CONR¹R², —S(O)ₙR¹, —NR²R³, —CH₂NR²R³, —OR¹, —CH₂OR¹ or —CO₂R⁴;
R¹, R² and R³ are independently selected at each occurrence from hydrogen, halogen, —C₁-C₄alkyl, aryl, heteroaryl, —C(O)R⁴, —C(O)NHR⁴, —CO₂R⁴ or —SO₂R⁴, or
R² and R³ in combination is —(CH₂)ⱼG(CH₂)ₖ— wherein G is oxygen, sulfur, NR⁴, or a bond;
j is 2, 3 or 4;
k is 0, 1 or2;
n is 0, 1 or 2, and
R⁴ is independently selected at each occurrence from hydrogen, —C₁-C₄alkyl, aryl, or heteroaryl, or a stereoisomer, enantiomer, in vivo-hydrolysable precursor or a pharmaceutically-acceptable salt thereof.

2. A compounds according to claim 1 wherein:
E is a 5-membered aromatic or heteroaromatic ring having 1 nitrogen atom and 1 oxygen atom, 1 oxygen atom, or 1 sulfur atom;
G is a 6-membered aromatic ring having 0 or 1 nitrogen atoms;
wherein G is unsubstituted or has 1 substituent selected from —C₁-C₆alkyl, —C₂-C₆alkenyl, —C₂-C₆alkynyl, halogen, —CN, —NO₂, —CF₃, —CONR¹R², —S(O)ₙR¹, —NR²R³, —CH₂NR²R³, —OR¹, —CH₂OR¹ or —CO₂R⁴, or a stereoisomer, enantiomer, in vivo-hydrolysable precursor or a pharmaceutically-acceptable salt thereof.

3. A compound according to claim 1 wherein:
E is a 5-membered aromatic or heteroaromatic ring having 1 nitrogen atom and 1 oxygen atom, 1 oxygen atom, or 1 sulfur atom;
G is a 6-membered aromatic ring having 0 or 1 nitrogen atoms;
wherein G is unsubstituted or has 1 substituent selected from —C₁-C₆alkyl, —CONR¹R² or —NR²R³, or a stereoisomer, enantiomer, in vivo-hydrolysable precursor or a pharmaceutically-acceptable salt thereof.

4. A compound according to claim 1 selected from:
3-(5-(5-phenylthiophen-2-yl)-1,3,4-oxadiazol-2-yl)quinuclidine;
3-(3-(5-phenylthiophen-2-yl)-1,2,4-oxadiazol-5-yl)quinuclidine;
3-[3-(5-Pyridin-3-yl-furan-2-yl)-[1,2,4]oxadiazol-5-yl]quinuclidine;
3-[3-(5-Pyridin-3-yl-thiophen-2-yl)-[1,2,4]oxadiazol-5-yl]quinuclidine;
3-[5-(5-Phenyl-furan-2-yl)-[1,3,4]oxadiazol-2-yl]quinuclidine;
3-[5-(5-Pyridin-3-yl-furan-2-yl)-[1,3,4]oxadiazol-2-yl]quinuclidine;
3-[5-(5-Pyridin-3-yl-thiophen-2-yl)-[1,2,4]oxadiazol-3-yl]quinuclidine;
3-[5-(5-Pyridin-3-yl-furan-2-yl)-[1,2,4]oxadiazol-3-yl]quinuclidine;
3-{5-[Quinuclidin-3-yl)-[1,2,4]oxadiazol-5-yl]-furan-2-yl}-N,N-dimethyl-benzamide;
3-{5-[5-(Quinuclidin-3-yl)-[1,3,4]oxadiazol-2-yl]-furan-2-yl}-N,N-dimethyl-benzamide;
3-[5-(5-Pyridin-3-yl-oxazol-2-yl)-[1,2,4]oxadiazol-3-yl]quinuclidine;
3-[5-(5-Pyridin-4-yl-oxazol-2-yl)-[1,2,4]oxadiazol-3-yl]quinuclidine;
3-(3-(5-(pyridin-3-yl)thiophen-2-yl)-1H-1,2,4-triazol-1-yl)quinuclidine, and
3-[3-(5-Phenyl-thiophen-2-yl)-[1,2,4]triazol-1-yl]quinuclidine.

5. A method of treatment or prophylaxis of a disease or condition in which activation of the α7 nicotinic receptor is beneficial which method comprises administering a therapeutically-effective amount of a compound according to claim 1 to a subject suffering from said disease or condition.

6. The method of claim 5, wherein said disease or condition is anxiety, schizophrenia, mania or manic depression.

7. A method of treatment or prophylaxis of neurological disorders, psychotic disorders or intellectual impairment disorders, which comprises administering a therapeutically effective amount of a compound according to claim 1.

8. The method of claim 7, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

9. A method for inducing the cessation of smoking comprising administering an effective amount of a compound according to claim 1.

10. A pharmaceutical composition comprising a compound according to claim 1 and a pharmaceutically-acceptable diluent, lubricant or carrier.

11. A method of treatment or prophylaxis of a disease or condition in which activation of the α7 nicotinic receptor is beneficial which method comprises administering a therapeutically-effective amount of a pharmaceutical composition according to claim 10 to a subject suffering from said disease or condition.

12. The method of claim 11, wherein said disease or condition is anxiety, schizophrenia, mania or manic depression.

13. A method of treatment or prophylaxis of neurological disorders, psychotic disorders or intellectual impairment disorders, which comprises administering a therapeutically effective amount of a pharmaceutical composition according to claim 10.

14. The method of claim 13, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

15. A method for inducing the cessation of smoking comprising administering an effective amount of a pharmaceutical composition according to claim 10.

16. The use of a compound according to claim 1, an enantiomer thereof or a pharmaceutically-acceptable salt thereof, in the manufacture of a medicament for the treatment or prophylaxis of human diseases or conditions in which activation of the α7 nicotinic receptor is beneficial selected from neurological disorders, psychotic disorders, intellectual impairment disorders, Alzheimer's disease, learning deficit, cognition deficit, attention deficit, memory loss, Attention Deficit Hyperactivity Disorder, anxiety, schizophrenia, mania or manic depression, Parkinson's disease, Huntington's disease, Tourette's syndrome, or neurodegenerative disorders in which there is loss of cholinergic synapses.

17. The use of a compound according to claim 1, in the manufacture of a medicament for the treatment or prophylaxis of jetlag, pain, or ulcerative colitis or to facilitate the cessation of smoking or the treatment of nicotine addiction or craving including that resulting from exposure to products containing nicotine.
